(19) United States
(12) Patent Application Publication
(10) Pub. No.: US 2005/0267365 A1
(43) Pub. Date: Dec. 1, 2005

(54) Method and apparatus for measuring anatomic structures

(76) Inventors: Alexander Sokulin, Kiriat Tivon (IL); Elina Burla, Kiriat Tivon (IL); Doron Hess, Haifa (IL)

Correspondence Address: John S. Beulick;Armstrong Teasdale LLP, Suite 2600, One Metropolitan Square, St. Louis, MO, 63102, US

(21) Appl. No.: 10/858,106

(22) Filed: Jun. 1, 2004

(51) Int. Cl. A61B 8/00

(52) U.S. Cl. 600/437

(57) ABSTRACT

A method for measuring an anatomic structure based on at least one medical diagnostic image frame using an integrated ultrasound device is provided. The image frame includes a first axis that is substantially parallel to an intima-media and a second axis that is perpendicular to the first axis. The method includes identifying a first interface of the anatomic structure based on an intensity of the interface in an image frame, for a plurality of points on the first interface, identifying a corresponding point on a second interface of the anatomic structure using a predetermined threshold based on the intensity of the first interface, determining a distance difference between points of the first interface and a corresponding point of the second interface, and outputting at least one of the determined distance difference and the at least one image frame to a display.

### BACKGROUND OF THE INVENTION

This invention relates generally to medical diagnostic systems. In particular, the present invention relates to methods and apparatus for acquiring and processing diagnostic data sets to identify the location of the transition between different types of tissue and between tissue and blood.

Coronary artery disease (CAD) has many known causes. The early detection and treatment of significant occlusive CAD before infarction is an important goal in reducing the downstream consequences of CAD. Many variables contribute to vascular health and may prove useful in the search for early markers of at-risk individuals. For example, echocardiography has the ability to measure one of the most important of the early markers, atherosclerotic burden. Atherosclerotic burden may be measured crudely during transesophageal echocardiography of the aorta. Further, as usually performed in clinical practice, the detection of plaque is qualitative at best, making it unlikely that robust data can be derived for early detection of preclinical artherosclerosis. Far more carefully studied is high resolution B-mode ultrasound scanning of the carotid arteries with measurement of intima-medial thickness (IMT). This test has been a mainstay of epidemiologic investigations of coronary and cerebrovascular disease for decades. Excellent data document the validity of using carotid findings to predict the state of the coronary circulation, and carotid IMT both detects patients with current disease as well as accurately predicting future cardiac and cerebrovascular events. Carotid IMT measurements have been proven to provide incremental data to traditional risk prediction based on clinical data. It is the only imaging test recommended by the American Heart Association for this purpose . Ultrasound imaging allows precise measurement of the total intima and media thickness of large- and medium-size peripheral arteries like the carotid, femoral, or radial arteries. The most common method to measure IMT is based on high-resolution B-mode imaging. Repeated and averaged manual measurement is relatively easy to perform, but is operator-dependent and of poor reproducibility. An accurate measurement with excellent reproducibility can be achieved only by using computer-assisted automatic methods.

Ultrasound imaging permits precise measurement of the total intima and media thickness of large and medium-size peripheral arteries, for example, the carotid, femoral, or radial arteries. At least some known methods to measure IMT are based on high-resolution B-mode imaging using repeated and averaged manual measurement. Manual measurement from B-mode images is relatively easy to perform, however, the results are operator-dependent and often of poor reproducibility. Other imaging modalities also may acquire vascular or cardiac images, but experience the same problems. In addition, it would be beneficial to more precisely identify and measure the interface between two types of tissue in other anatomy or masses of interest, such as the liver, heart, cysts and tumors.

### BRIEF DESCRIPTION OF THE INVENTION

In one embodiment, a method for measuring an anatomic structure based on at least one medical diagnostic image frame using an integrated ultrasound device is provided. The image frame includes a first axis that is substantially parallel to an intima-media and a second axis that is perpendicular to the first axis. The method includes identifying a first interface of the anatomic structure based on an intensity of the interface in an image frame, for a plurality of points on the first interface, identifying a corresponding point on a second interface of the anatomic structure using a predetermined threshold based on the intensity of the first interface, determining a distance difference between points of the first interface and a corresponding point of the second interface, and outputting at least one of the determined distance difference and the at least one image frame to a display.

In another embodiment, an integrated ultrasound device is provided. The device includes a transmitter for transmitting ultrasound signals into an area of interest, a receiver for receiving echo signals from transmitted ultrasound signals, a memory for storing at least one image frame including the echo signals, an electro-cardiograph gating (ECG) waveform and synchronization unit, a processor configured to process the at least one image frame to automatically identify at least one of a lumen-intima interface and a media adventia interface, and an output for outputting information based on an output of the processor.

In a further embodiment, a computer program embodied on a computer readable medium for controlling an integrated ultrasound device is provided. The program controls the integrated ultrasound device to measure an intima-media thickness (IMT) based on at least one medical diagnostic image frame that includes a first axis that is substantially parallel to the intima-media and a second axis that is perpendicular to the first axis. The program includes a code segment that prompts a user for at least one of the image frame and a selected region of interest, and then determines a first axis and second axis coordinate for a plurality of points on a media adventia interface, determines a first axis and second axis coordinate for a plurality of points on a lumen-intima interface, determines a distance difference between a point on the media adventia interface and a corresponding point on the lumen-intima interface, and outputs a statistical analysis of at least one of the plurality of points, the output includes at least one of an average IMT, an IMT standard deviation, an IMT maximal value, and an IMT minimal value.

### BRIEF DESCRIPTION OF THE DRAWINGS

FIG. 1 is a block diagram of an ultrasound system constructed in accordance with an embodiment of the present invention;

FIG. 2 is an ultrasound system constructed in accordance with another embodiment of the present invention;

FIG. 3 is a schematic view of a longitudinal cross-section of an exemplary artery that may be scanned using the ultrasound system shown in FIG. 1; and

FIG. 4 is a flow chart of an exemplary method for automatic detection of lumen-intima and media-adventitia interfaces that may be used with the ultrasound system shown in FIG. 1.

### DETAILED DESCRIPTION OF THE INVENTION

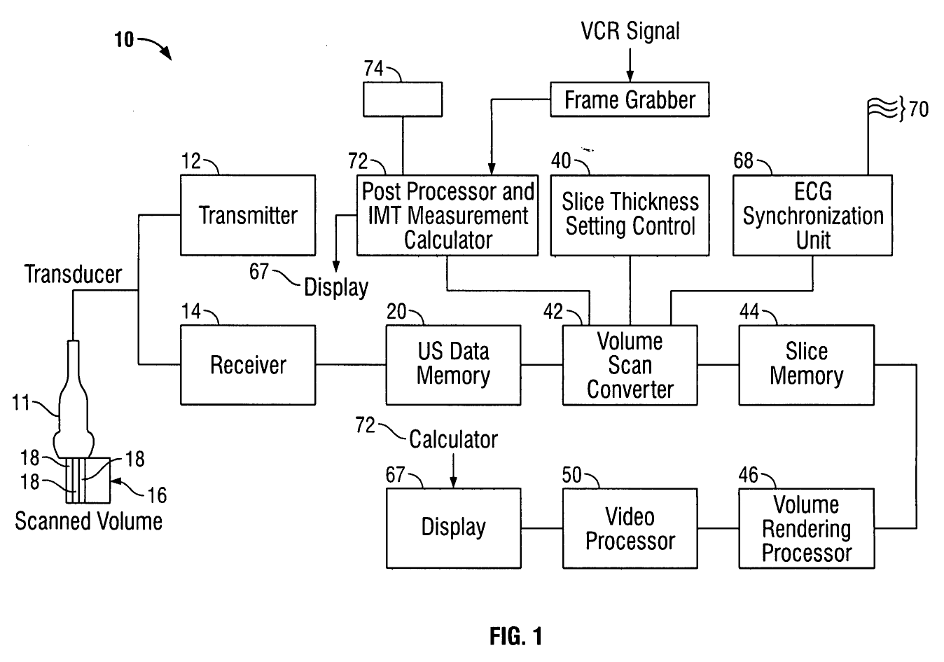

FIG. 1 illustrates an ultrasound system 10 constructed in accordance with one embodiment of the present invention. The system includes a transducer 11 connected to a transmitter 12 and a receiver 14. The transducer 11 transmits ultrasonic pulses and receives echoes from structures inside of a scanned ultrasound image or volume 16. Memory 20 stores ultrasound data from the receiver 14 derived from the scanned ultrasound image or volume 16. The image or volume 16 may be obtained by various techniques (e.g., 3D scanning, real-time 3D imaging, volume scanning, 2D scanning with transducers having positioning sensors, freehand scanning using a Voxel correlation technique, 2D or matrix array transducers and the like).

Transducer 11 may be moved, such as along a linear or arcuate path, while scanning a region of interest (ROI). The scan planes 18 are stored in the memory 20, and then passed to a scan converter 42. Scan-converter 42 synchronizes the modules of ultrasound system 10. In some embodiments, the transducer 11 may obtain lines instead of the scan planes 18, and the memory 20 may store lines obtained by the transducer 11 rather than the scan planes 18. The scan converter 42 may store lines obtained by the transducer 11 rather than the scan planes 18. The scan converter 42 creates a data slice from a single scan plane 18. The data slice is stored in slice memory 44 and then passed to the video processor 50 and display 67. System 10 may facilitate measuring an anatomic structure within the region of interest in at least one of a real-time mode, a frame freeze mode, a cine-loop run mode, a VCR playback mode, and an ultrasound device internal archive single frame or loop frame playback mode.

An integral ECG waveform and synchronization unit 68 is coupled to a patient skin (not shown). ECG waveform and synchronization unit 68 uses a plurality of electrodes 70 to measure electrical current passing through a patient's body. The electrical current corresponds to the electrical activity of the patient's heart muscles, or the contraction and relaxation thereof. This current may be used to identify a cyclical portion of the heart's cycle, thus allowing blood-vessel data to be acquired during intervals that substantially correspond to a substantially similar portion of the heart's cycle when the blood vessel is in a substantially uniform position. A post-processor and intima-media thickness measurement calculator 72 may receive raw image data and/or scan converted data to identify structures of a blood vessel and automatically determine thicknesses of those structures. Post-processor and intima-media thickness measurement calculator 72 may also receive archive data from a data archive 74, such as a video cassette recorder (VCR) and/or a VCR play-back internal frame grabber unit, or other data storage device, which may be located locally to system 10, integral with system 10, or may be located remotely from system 10 and accessed over a data network (not shown). Post-processor and intima-media thickness measurement calculator 72 may transmit highlighting signals to display 67 to provide trace determined interfaces with a brighter pixel intensity and/or false color highlight of determined interfaces to aid an operator in determining accurate output.

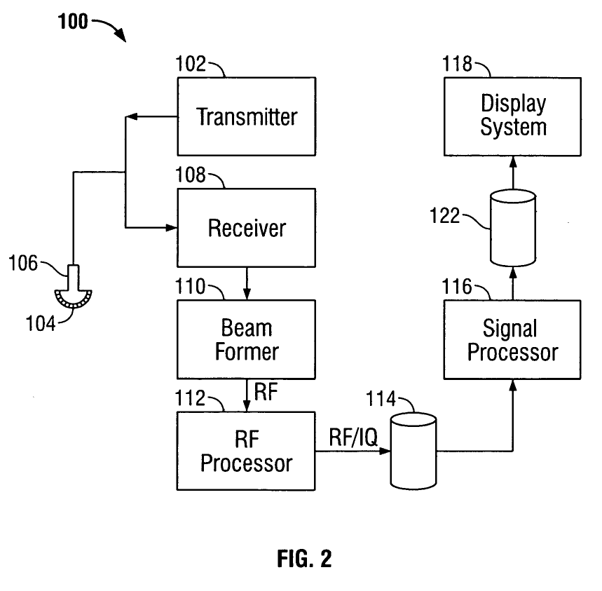

FIG. 2 illustrates a block diagram of an ultrasound system 100 constructed in accordance with another embodiment of the present invention. The ultrasound system 100 includes a transmitter 102, which drives an array of elements 104 within a transducer 106 to emit pulsed ultrasonic signals into a body. A variety of geometries may be used. The ultrasonic signals are back-scattered from structures in the body, like blood cells or muscular tissue, to produce echoes, which return to the elements 104. The echoes are received by a receiver 108. The received echoes are passed through a beamformer 110, which performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to RF/IQ buffer 114 for temporary storage.

The ultrasound system 100 also includes a signal processor 116 to process the acquired ultrasound information (i.e., RF signal data or IQ data pairs) and prepare frames of ultrasound information for display on display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally, or alternatively, the ultrasound information may be stored temporarily in RF/IQ buffer 114 during a scanning session and processed in less than real-time in a live or off-line operation.

The ultrasound system 100 may continuously acquire ultrasound information at a frame rate that exceeds fifty frames per second, which is the approximate perception rate of the human eye. The acquired ultrasound information may be displayed on the display system 118 at a slower frame-rate. An image buffer 122 is included for storing processed frames of acquired ultrasound information that are not scheduled to be displayed immediately. The image buffer 122 may be of sufficient capacity to store at least several seconds worth of frames of ultrasound information. The frames of ultrasound information are stored in a manner to facilitate retrieval thereof according to its order or time of acquisition. The image buffer 122 may comprise any known data storage medium.

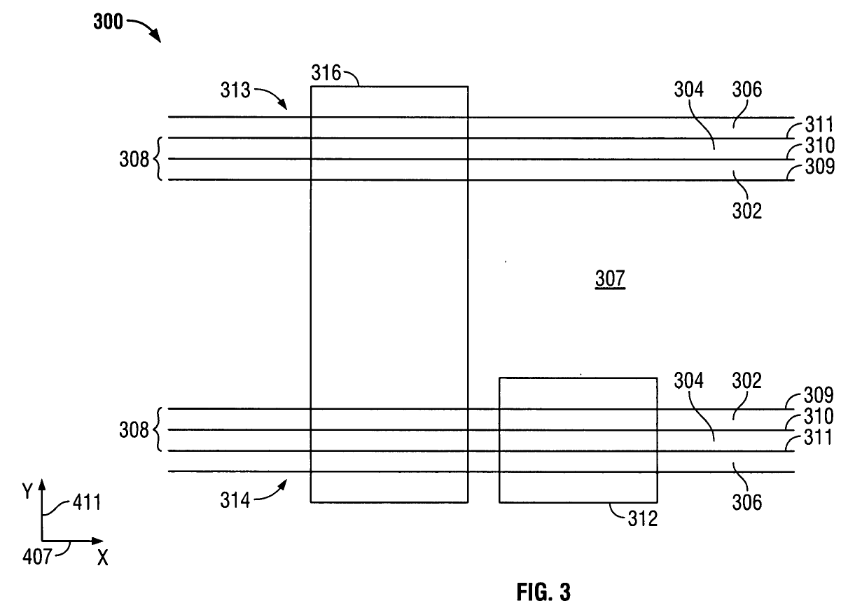

FIG. 3 illustrates a view of a longitudinal cross-section of an exemplary artery 300 that may be scanned using system 10 shown in FIG. 1. In the exemplary embodiment, artery 300 includes three layers, a tunica intima 302, a media 304, and adventitia 306 that define a tubular cavity therein referred to as a lumen 307. At least some known diseases may cause one or more of such layers to thicken and/or obtain a plaque coating. For example, tunica intima 302 or media 304 may thicken due to a lesion. Specifically, arterial sclerosis may cause intima 302 to thicken. Further, hypertension may cause media 304 to thicken. Accordingly, respective diseases may be evaluated by measuring the thickness of intima 302, the thickness of media 304 or a combined measurement of an intima-medial thickness (IMT) 308. To facilitate measuring thicknesses of the various layers, an interface between the layers may be used, such as a lumen-intima interface 309, a intima-media interface 310, and a media-adventitia interface 311.

During a scan, a user may select a region of interest (ROI) 312 that includes a portion of an anterior wall 313 and/or a portion of a posterior wall 314 of a vessel, such as artery 300. A user may select a second ROI 316 that includes both a portion of anterior wall 313 and a portion of posterior wall 314 of the vessel, if the scan is to be performed using tissue motion synchronization. Tissue motion synchronization and electro-cardiograph gating (ECG) synchronization permits evaluation of frames captured at a fixed time relative to an oscillatory motion of a tissue, such as, a vein or artery wall, and heart wall, at a fixed time relative to a heart cycle. In the exemplary embodiment, the IMT measurement is performed at a selectable fixed point in time relative to the ECG waveform, for example, during end-diastole. System 10 can synchronize on any part of the ECG waveform received from the built-in ECG unit or from an external ECG waveform. The synchronization location is preselectable and automatic, but may be manually adjusted.

The synchronization discussed above may be selected by a user to be based on vessel-wall motion detection rather than on ECG waveform. The same IMT algorithm is used, in this case, to delineate both walls of artery 300 (anterior 313 and posterior 314). By measuring the distance between anterior wall 313 and posterior wall 314 as function of time, it is possible to follow the pulsatility of artery 300, and synchronize the frame-selection for an IMT measurement similarly to the synchronization to the ECG waveform.

System 10 may receive image data at a resolution greater than display is capable of displaying. For example system 10 may be able to obtain images with a 1200 pixel per inch resolution, but display 67 may only be capable of displaying 400 pixels per inch. System 10 may use the display resolution images to measure IMT and may selectably zoom display 67 to use the entire 1200 pixel per inch resolution available in the received image. Such a zoom feature facilitates an accurate IMT measurement. System 10 also configured to detect a zoom setting for each frame of image data, for example, live data, archived data, and frame grabbed data from a VCR playback. A user may retrieve image frame data from a plurality of image frame data sources, such as real-time data raw data, real-time data preprocessed data, frame freeze data, cine-loop data, and/or VCR playback data. When reviewing image frame data from various sources collected at widely varying timeframes, each image frame may be stored in different resolution setting with respect to each other image frame. Correlating image frames over time may be used while making a diagnosis, such that viewing image frames at different resolution settings may cause errors to be made. System 10 may selectably read the resolution setting and zoom setting of each image frame and automatically modify the resolution setting and/or zoom setting of each image frame to be consistent with respect to each other at a user-preferred selectable setting.

To facilitate imaging and the determination of the vessel IMT a contrast agent may be injected into the vessel prior to or during a scan. Generally, contrast is injected into the blood-stream passing through the vessel, such as artery 300, to facilitate enhancing visibility of the blood vessels and the vessel-borders delineation. The contrast agent also may enhance the visibility and delineation of “soft-plaque”, which may be non-reflective (i.e., has very dark gray shade) and may be difficult to distinguish from the surrounding blood filled lumen. The contrast agent facilitates sound reflection of the blood, such that the blood appears as a lighter gray shade and the relatively darker soft-plaque becomes more distinguishable and visible in the ultrasound image.

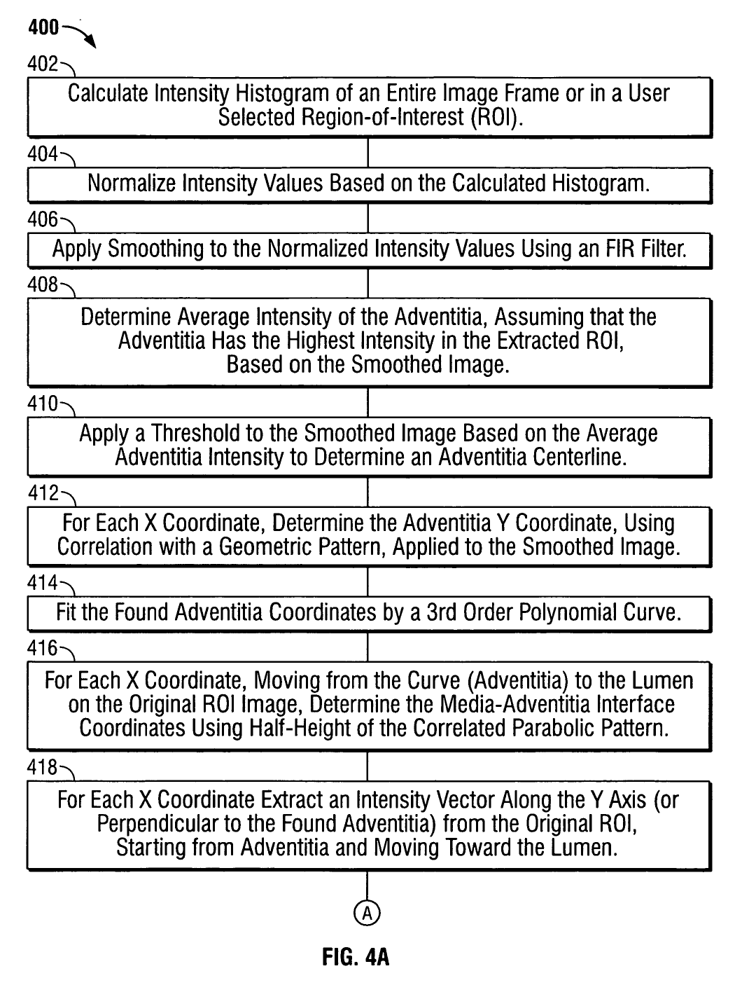

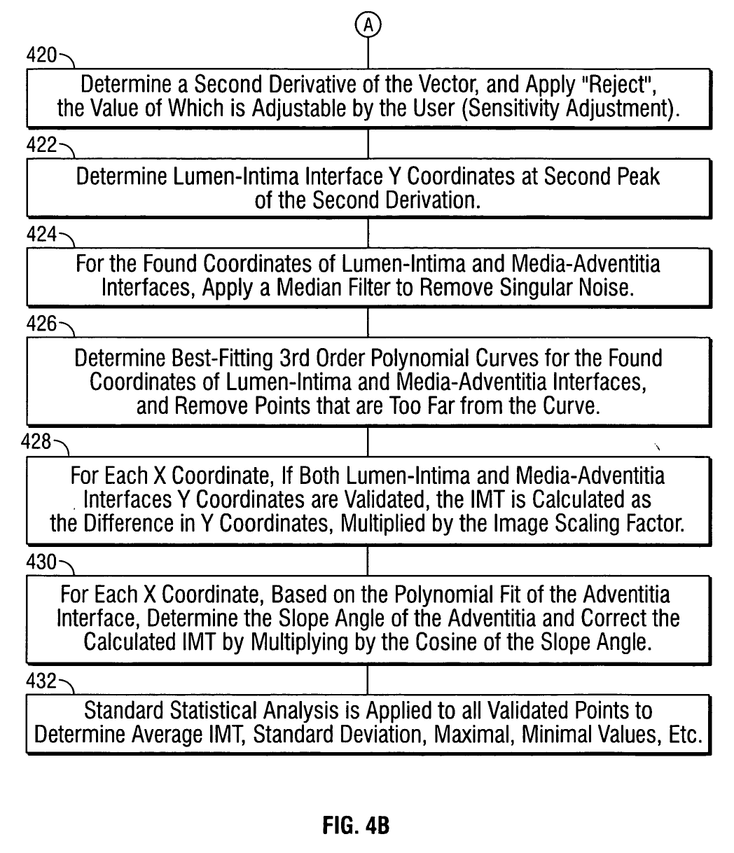

FIG. 4 illustrates an exemplary method 400 for automatic detection of lumen-intima and media-adventitia interfaces. A process or algorithm of method 400 uses a gray-scale image produced from raw ultrasound data by scan-conversion, or can be applied to raw data before scan-conversion, or may be applied to any pixel-data image, for example, a pixel-data image that includes a visible double-line intima-media pattern.

Method 400 includes calculating 402 an intensity histogram of an entire image frame or in a user selected region-of-interest (ROI). A user selected ROI should include a part of lumen 307 and adventitia 306 (shown in FIG. 3). Image frame intensity values may be normalized 404 based on the calculated histogram. Smoothing using, for example, a finite impulse response (FIR) filter is applied 406 laterally along the image in a direction of artery 300 (shown in FIG. 3), for example, an x-axis 407 that may be selected relative to an orientation of artery 300. The orientation of x-axis 407 may be independent of the orientation of artery 300 as viewed in display 67. In the exemplary embodiment, the orientation of artery 300 corresponds to a horizontal x-axis 407. In other embodiments, artery 300 may be oriented in any direction with respect to artery 300. Method 400 then determines 408 an average intensity of adventitia 306, assuming that adventitia 306 has the highest intensity in the image frame or in the extracted ROI 312 or 316, based on the smoothed image. A threshold is applied 410 to the smoothed image based on the average adventitia intensity, determined at 408 to determine a centerline of the adventitia. For each x-axis 407 coordinate of the adventitia centerline, a y-axis 411 coordinate of the adventitia centerline is determined using a correlation with a geometric pattern, such as a parabolic pattern or a cosine pattern, applied to the smoothed image. The determined adventitia x-axis 407 and y-axis 411 coordinates are then fit 414 by a third order polynomial curve.

For each x-axis 407 coordinate, moving from the curve (adventitia 306) to lumen 307, the coordinates of media-adventitia interface 311 are determined 416 using a half-height of the correlated geometric pattern. For each x-axis 407 coordinate, an intensity vector along y-axis 411, for example, perpendicular to the determined adventitia, is determined from original ROI 312 or 316, starting from adventitia 306 and moving toward lumen 307. A second derivative of the vector is determined 420, and a threshold is applied to further delineate the interfaces. The value of the applied threshold is adjustable by the user as a sensitivity adjustment. Lumen-intima interface y-axis 411 coordinates at a second peak of the second derivation is determined 422. For the determined coordinates of lumen-intima interface 309 and media-adventitia interface 311, a median filter is applied 424 to remove singular noise. Best-fitting third order polynomial curves for the determined coordinates of lumen-intima interface 309 and media-adventitia interface 311 are determined, and the interface points are validated by removing 426 points that exceed a predetermined distance from the best fit curve.

For each x-axis 407 coordinate, if both lumen-intima interface 309 and media-adventitia interface 311 y-axis 411 coordinates are validated, the IMT is calculated as the difference between y-axis 411 coordinates of the lumen-intima interface point and media-adventitia interface point at that x-axis 407 coordinate. The difference may then be multiplied by an image-scaling factor. For each x-axis 407 coordinate, based on the polynomial fit of the adventitia interface, the slope angle of the adventitia with respect to lumen-intima interface 309 is determined 430 and the calculated IMT is corrected by multiplying by the cosine of the slope angle. Apply 432 standard statistical analysis to all validated points to determine, for example, but not limited to an average IMT, an IMT standard deviation, an IMT maximal value, and an IMT minimal value.

Method 400 may include injection of a contrast agent into the blood vessel to facilitate enhancing visibility and delineation of the vessel walls. When using a contrast agent, a plurality of initialization parameters may be modified or additional parameters set to indicate to the IMT algorithm that a contrast agent is being used and characteristic is relative to the particular contrast agent used.

A technical effect of various embodiments of the present invention is to automatically identify and measure an anatomical structure. Specifically, the system captures image frames of ultrasound data representing a region of interest. In one embodiment of the present invention, structures of a blood vessel are located, identified, and measured. Various methods of displaying the output of the structure and measurements are selectable to facilitate diagnosis.

While various embodiments the present invention have been described with reference to an integrated ultrasound scanner configured to automatically measure IMT in a blood vessel, numerous other applications are contemplated. It is contemplated that the method and systems of the present invention may be applied to other imaging modalities, such as MRI, and anatomic structure other than a blood vessel.

The above-described systems and methods of automatically measuring IMT in a blood vessel in an integrated ultrasound scanner are cost-effective and highly reliable for facilitating monitoring and diagnosing disease. More particularly, the methods and systems described herein facilitate identifying and determining a thickness of, for example, blood vessels in an integrated ultrasound scanner. As a result, the methods and systems described herein facilitate reducing healthcare costs in a cost-effective and reliable manner.

Exemplary embodiments of real-time integrated ultrasound systems and methods are described above in detail. However, the systems are not limited to the specific embodiments described herein, but rather, components of each system may be utilized independently and separately from other components described herein. Each system component also can be used in combination with other system components.

While the invention has been described in terms of various specific embodiments, those skilled in the art will recognize that the invention can be practiced with modification within the spirit and scope of the claims.

1. A method for measuring a thickness of a portion of a multi-layer vessel based on at least one medical diagnostic image frame using an integrated ultrasound device, the at least one image frame including a first axis that is substantially parallel to an intima-media of the anatomic structure and a second axis that is perpendicular to the first axis, said method comprising:
identifying a centerline of one layer of the multi-layers of the vessel based on an intensity of the layer in an image frame;
determining a first interface of the first layer;
identifying, for a plurality of points on the first interface, a corresponding point on a second interface;
determining a distance difference between the plurality of points on the first interface and a corresponding point on the second interface; and
outputting at least one of the determined distance difference and the at least one image frame to a display.

2. A method in accordance with claim 1 further comprising performing at least one of a intima-media thickness (IMT) measurement, an abdominal scan, an OB/GYN scan, surgical monitoring, and a cardiac, a vascular and a pediatric scan.

3. A method in accordance with claim 1 further comprising;
storing the at least one image frame to at least one of an internal image archive unit and a VCR play-back internal frame grabber unit; and
retrieving the at least one image frame from at least one of an internal image archive unit and a VCR play-back internal frame grabber unit.

4. A method in accordance with claim 1 further comprising:
coupling an ECG waveform and synchronization unit to a patient wherein the ECG waveform and synchronization unit is integral to the integrated ultrasound device; and
transmitting heart cycle information from the patient to a processor of the integrated ultrasound device.

5. A method in accordance with claim 4 further comprising automatically synchronizing the at least one image frame with a selectable portion of at least one of said heart cycle information and an external ECG waveform.

6. A method in accordance with claim 1 wherein the first interface is a lumen-intima interface and the second interface is a media-adventia interface, said method further comprising highlighting at least one of a lumen-intima interface and a media-adventia interface.

7. A method in accordance with claim 6 further comprising altering the resolution of the at least one image frame to facilitate measuring the IMT of the multi-layers of the vessel.

8. A method in accordance with claim 6 further comprising:
calculating an intensity histogram of a region of interest of the at least one image;
determining an adventitia using the intensity histogram and an average intensity of the adventitia calculated using the intensity histogram;
highlighting a media-adventia interface based on a boundary of the determined adventitia;
determining a second axis value of the lumen-intima interface at each value of the first axis of the adventia; and
highlighting the lumen-intima interface.

9. A method in accordance with claim 6 further comprising determining an IMT measurement value using the difference of the second axis coordinate values of the media-adventia interface and the lumen-intima interface at each first axis coordinate.

10. A method in accordance with claim 9 further comprising correcting the IMT measurement value based on a slope of the media-adventia interface.

11. A method in accordance with claim 1 further comprising measuring an anatomic structure in at least one of a real-time mode, a frame freeze mode, a cine-loop run mode, a VCR playback mode, and an ultrasound device internal archive single frame or loop frame playback mode.

12. A method in accordance with claim 1 further comprising processing at least one of raw data ultrasound image frames and preprocessed ultrasound image frames.

13. A method in accordance with claim 1 further comprising displaying an IMT measurement output that includes at least one of a tracing of the lumen-intima interface, a tracing of the media-adventitia interface, an average IMT value based upon a selectable plurality of determined lumen-intima interface points and media-adventitia interface points, a standard deviation of an IMT value measurement, an IMT value measurement maximal point, an IMT value measurement minimal point, a graph of the IMT value measurement as function of location along a vessel, a graph of the IMT value measurement as function of time inside a heart cycle, and derived statistical values for the displayed lumen-intima interface and/or the media-adventitia interface curves.

14. A method in accordance with claim 13 further comprising displaying at least one image frame concurrently with at least one respective IMT measurement on the same display.

15. A method in accordance with claim 1 further comprising:
prompting a user for a signal indicative of approval or disapproval of the output; and
if the signal indicates approval, saving the output of the processor to an integrated ultrasound device archiving unit.

16. A method in accordance with claim 1 wherein the first interface comprises a lumen-intima interface and the second interface comprises a media-adventia interface, and wherein said method further comprises identifying at least one of the lumen-intima interface and the media adventia interface based on a single frame, averaged over a plurality of frames, and averaged over a plurality of frames synchronized at a selectable specific heart-cycle point.

17. A method in accordance with claim 1 wherein outputting at least one of the determined distance difference and the at least one image frame to a display comprises outputting automatic image display calibration information for the at least one image frame.

18. A method in accordance with claim 1 further comprising receiving a selectable sensitivity adjustment signal wherein the sensitivity adjustment signal facilitates reducing at least one of circuit noise, soft plaque response, artifact response, and soft tissue response.

19. A method in accordance with claim 1 further comprising receiving a selectable sensitivity adjustment signal to delineate one or more plaque borders for measuring a plaque thickness.

20. A method in accordance with claim 1 further comprising:
automatically identifying at least one of an anterior lumen-intima interface, an anterior media adventia interface, a posterior lumen-intima interface, and a posterior media adventia interface;
determining a distance between at least one of the anterior lumen-intima interface and the anterior media adventia interface, and the posterior lumen-intima interface and the posterior media adventia interface during a selectable portion of a heart cycle; and
automatically synchronizing image frame selection with the a pre-determined time-slot of the heart cycle based on a delay from a synchronization point on the ECG waveform relative to the determined distance.

21. A method in accordance with claim 1 further comprising:
measuring an IMT for a plurality of image frames representing a plurality of different portions of a heart cycle;
displaying a set of IMT measurements for a selectable plurality of different portions of a heart cycle; and
automatically determining at least one of a contractility and an elasticity of at least one of a vessel wall and a layer of plaque.

22. A method in accordance with claim 1 further comprising:
measuring an IMT for a plurality of image frames representing a plurality of substantially identical selectable portions of a heart cycle; and
displaying an average of a set of IMT measurements for the selected plurality of substantially identical portions of the heart cycle.

23. A method in accordance with claim 1 further comprising:
retrieving a plurality of archived image frames from different scans taken over a predetermined period of time;
determining a zoom setting of each image frame;
modifying a zoom setting of each image frame to a user-preferred selectable setting; and
measuring a change in the IMT of the vessel over the period of time.

24. A method in accordance with claim 1 further comprising:
administering a contrast agent to the vessel to be imaged; and
modifying at least one of an initialization parameter indicative of the contrast agent used.

25. An integrated ultrasound device comprising:
a transmitter for transmitting ultrasound signals into an area of interest;
a receiver for receiving echo signals from the transmitted ultrasound signals;
a memory for storing at least one image frame including the echo signals;
an electrocardiograph gating (ECG) waveform and synchronization unit;
a processor configured to process said at least one image frame to automatically identify at least one of a lumen-intima interface and a media adventia interface of a vessel wall; and
an output for outputting information based on an output of said processor.

26. An integrated ultrasound device in accordance with claim 25 wherein the processor is further configured to control at least one of a intima-media thickness (IMT) measurement, an abdominal scan, an OB/GYN scan, surgical monitoring, a cardiac scan, a vascular scan, and a pediatric scan.

27. An integrated ultrasound device in accordance with claim 25 further comprising at least one of an internal image archive unit and a VCR play-back internal frame grabber unit.

28. An integrated ultrasound device in accordance with claim 25 wherein said ECG waveform and synchronization unit is configured to couple to a patient and to transmit heart cycle information from said patient to said processor.

29. An integrated ultrasound device in accordance with claim 28 wherein said processor is further configured to automatically synchronize image frame selection with a selectable portion of at least one of said heart cycle information and an external ECG waveform.

30. An integrated ultrasound device in accordance with claim 25 wherein said processor is further configured to highlight at least one of a lumen-intima interface and a media-adventia interface.

31. An integrated ultrasound device in accordance with claim 30 wherein said processor is further configured to:
calculate an intensity histogram of a region of interest of the at least one image wherein said image is referenced to a Cartesian coordinate system;
determine an adventitia centerline using said intensity histogram and an average intensity of the adventitia calculated using said intensity histogram;
highlight a media-adventia interface based on a boundary of the determined adventitia;
determine a second axis value of the lumen-intima interface corresponding to a first axis value of the adventia; and
highlight the lumen-intima interface.

32. An integrated ultrasound device in accordance with claim 30 wherein said processor is further configured to determine an IMT measurement value using the difference of the second axis coordinate values of the media-adventia interface and the lumen-intima interface at each first axis coordinate.

33. An integrated ultrasound device in accordance with claim 32 wherein said processor is further configured to correct the IMT measurement value based on a slope of the media-adventia interface.

34. An integrated ultrasound device in accordance with claim 25 wherein said processor is further configured to process said at least one image frame in at least one of a real-time mode, a frame freeze mode, a cine-loop run mode, a VCR playback mode, and an ultrasound device internal archive single frame or loop frame playback mode.

35. An integrated ultrasound device in accordance with claim 25 wherein said processor is configured to process at least one of raw data ultrasound image frames and preprocessed ultrasound image frames.

36. An integrated ultrasound device in accordance with claim 25 wherein said output is configured to display IMT measurements comprising at least one of a tracing of the lumen-intima interface, a tracing of the media-adventitia interface, an average IMT value based upon a selectable plurality of determined lumen-intima interface points and media-adventitia interface points, a standard deviation of an IMT value measurement, an IMT value measurement maximal point, an IMT value measurement minimal point, a graph of the IMT value measurement as function of location along a vessel, a graph of the IMT value measurement as function of time inside a heart cycle, and derived statistical values for the at least one displayed lumen-intima interface and the media-adventitia interface curves.

37. An integrated ultrasound device in accordance with claim 36 wherein said output is configured to display said at least one image frame concurrently with said IMT measurements.

38. An integrated ultrasound device in accordance with claim 25 wherein said processor is further configured to:
prompt a user for a signal indicative of approval or disapproval of the output; and
if the signal indicates approval, the output of said processor is saved to an integrated ultrasound device archiving unit.

39. An integrated ultrasound device in accordance with claim 25 wherein said processor is further configured to identify said at least one of a lumen-intima interface and a media adventia interface based on a single frame, averaged over a plurality of frames, and averaged over a plurality of frames synchronized at a selectable specific heart-cycle point.

40. An integrated ultrasound device in accordance with claim 25 wherein said output includes automatic image display calibration information.

41. An integrated ultrasound device in accordance with claim 25 wherein said processor is further configured to receive a selectable sensitivity adjustment signal, said signal used to facilitate reducing at least one of circuit noise, soft plaque response, artifact response, and soft tissue response.

42. An integrated ultrasound device in accordance with claim 25 wherein said processor is further configured to receive a selectable sensitivity adjustment signal to delineate one or more plaque borders for measuring a plaque thickness.

43. An integrated ultrasound device in accordance with claim 25 wherein said processor is further configured to:
automatically identify an anterior at least one of a lumen-intima interface and a media adventia interface, and a posterior at least one of a lumen-intima interface and a media adventia interface;
determine a distance between said identified interfaces during a heart cycle; and
automatically synchronize an image frame selection with the a pre-determined time-slot of the heart cycle based on a delay from a synchronization point on the ECG waveform relative to the determined distance.

44. An integrated ultrasound device in accordance with claim 25 wherein said processor is further configured to:
measure an IMT for a plurality of image frames representing a plurality of different portions of a heart cycle;
display a set of IMT measurements for a selectable plurality of different portions of a heart cycle; and
automatically determine at least one of a contractility and an elasticity of at least one of the vessel wall and a layer of plaque.

45. An integrated ultrasound device in accordance with claim 25 wherein said processor is further configured to:
measure an IMT for a plurality of image frames representing a plurality of substantially identical selectable portions of a heart cycle; and
display an average of a set of IMT measurements for the selected plurality of substantially identical portions of the heart cycle.

46. An integrated ultrasound device in accordance with claim 25 wherein said processor is further configured to:
receive an input relative to a contrast agent being used during the scan; and
modify at least one of an initialization parameter indicative of the contrast agent used.

47. A computer program embodied on a computer readable medium for controlling an integrated ultrasound device to measure an intima-media thickness (IMT) based on at least one medical diagnostic image frame comprising a first axis that is substantially parallel to the intima-media and a second axis that is perpendicular to the first axis, said program comprising a code segment that prompts a user for at least one of the image frame and a selected region of interest, and then executes instructions that:
determines a first axis and second axis coordinate for a plurality of points on a media adventia interface;
determines a first axis and second axis coordinate for a plurality of points on a lumen-intima interface;
determines a distance difference between a point on the media adventia interface and a corresponding point on the lumen-intima interface; and
outputs a statistical analysis of at least one of the plurality of points, the output includes at least one of an average IMT, an IMT standard deviation, an IMT maximal value, and an IMT minimal value.

48. A computer program in accordance with claim 47 wherein said code segment that determines a first axis and second axis coordinate for a plurality of points on a media adventia interface further comprises a code segment that:
calculates an intensity histogram of the at least one of the image frame and the selected region-of-interest (ROI);
normalizes the intensity values based on the calculated histogram;
applies lateral smoothing using a finite impulse response (FIR) filter;
identifies an adventitia based on the intensity of the smoothed image;
determines an average intensity of the adventitia;
applies a threshold to the smoothed image based on the determined average adventitia intensity;
for each first axis coordinate of the adventitia, determines an adventitia second axis coordinate using a correlation with at least one of a parabolic pattern and a cosine pattern applied to the smoothed image; and
fits the determined adventitia first and second axis coordinates to a third order polynomial curve.

49. A computer program in accordance with claim 47 wherein said code segment that determines a first axis and second axis coordinate for a plurality of points on a lumen-intima interface further comprises a code segment that:
for each first axis coordinate of the adventitia, stepping from the adventitia curve toward the lumen, determines the media-adventitia interface coordinates using a half-height of the correlated parabolic pattern;
for each first axis coordinate of the adventitia, determines an intensity vector along the second axis starting from the adventitia and stepping toward the lumen;
determines a second derivative of the vector, and applies a user selectable threshold;
determines a lumen-intima interface second axis coordinate at second peak of the second derivation;
for the identified coordinates of lumen-intima and media-adventitia interfaces, applies a median filter to remove singular noise; and
determines best-fitting third order polynomial curves for the identified coordinates of lumen-intima and media-adventitia interfaces, and validates points that are within a selectable threshold distance from the determined curves.

50. A computer program in accordance with claim 47 wherein said code segment that determines a distance difference between a point on the media adventia interface and a corresponding point on the lumen-intima interface further comprises a code segment that:
calculates the IMT using a difference in respective second axis coordinates, multiplied by an image scaling factor for each first axis coordinate if both lumen-intima and media-adventitia interfaces second axis coordinates are validated; and
determines a slope angle of the adventitia at each first axis coordinate and corrects the calculated IMT by multiplying by the cosine of the slope angle for each first axis coordinate, based on the polynomial fit of the adventitia interface.
